(19) United States
(12) Patent Application Publication
(10) Pub. No.: US 2007/0270806 A1
(43) Pub. Date: Nov. 22, 2007

(54) Devices and methods for receiving elongated connecting elements in spinal surgical procedures

(76) Inventors: Kevin T. Foley, Germantown, TN (US); Anthony J. Melkent, Memphis, TN (US)

Correspondence Address: KRIEG DEVAULT LLP, ONE INDIANA SQUARE, SUITE 2800, INDIANAPOLIS, IN, 46204-2709, US

(21) Appl. No.: 11/400,166

(22) Filed: Apr. 7, 2006

(51) Int. Cl. A61F 2/30

(52) U.S. Cl. 606/61

(57) ABSTRACT

Devices and methods include an anchor assembly engageable to a vertebra and a connecting element positionable through a receiver of the anchor assembly. The receiver includes chamfered surfaces along a leading end of the receiver to facilitate endwise insertion of the connecting element into the passage.

### BACKGROUND

Elongated connecting elements, such as rods, plates, tethers, wires, cables, and other devices have been implanted along the spinal column and connected between two or more anchors engaged between one or more spinal motion segments. Such connecting elements can be positioned in the anchor with a top-down approach or a side-to-side approach. In the top-down approach, the incision extends between the anchors, and the connecting element is moved distally or toward the opposite side of the patient through the incision until it is positioned for engagement to the anchors. For example, in posterior spinal surgery, the connecting element is moved anteriorly through a posterior incision to the anchors. In the side-to-side approach, the connecting element is positioned distally through incision to a location adjacent the anchors, and then moved in a sideways direction until it is positioned for engagement with the anchors. For example, in posterior spinal surgery, the connecting element is moved anteriorly through a posterior incision to a location adjacent the anchor, and then moved medially or laterally, depending on the relative anchor location, to the anchor engaging position.

Other surgical instruments and techniques contemplate insertion of a connecting element to an anchor engaging position along a path that extends along or is generally parallel an anchor alignment axis extending between the anchors. Examples of such instruments and techniques are provided in U.S. Pat. No. 6,530,929, which is incorporated herein by reference in its entirety.

### SUMMARY

The present invention generally relates to devices and methods that facilitate connecting element insertion along or generally parallel to the anchor alignment axis in spinal surgical procedures.

In one form, an anchor assembly includes a connecting element receiving portion defining a passage between a pair of arms. The arms include opposite end surfaces at leading and trailing ends thereof. At the leading ends of the arms include a chamfered surface extending from the passage and a leading end surface generally orthogonal to the connecting element insertion axis extending through the passage. As used herein, the leading end of the receiver refers to the end of the receiver oriented in the direction from which the connecting element is positioned into the passage in an endwise fashion.

In another form, a spinal surgical method includes engaging an anchor assembly to at least one vertebra; orienting a receiving portion of the anchor assembly so that a passage defined by the receiving portion is oriented along a connecting element insertion axis; and inserting a connecting element along the connecting element insertion axis and into the passage of the receiving portion, wherein the receiving portion includes a chamfered surface extending from the passage to a leading end surface of the receiving portion, the leading end surface being transversely oriented to the connecting element insertion axis.

These and other aspects will be discussed further below.

### BRIEF DESCRIPTION OF THE DRAWINGS

FIG. 1 is a perspective view of an anchor assembly.

FIG. 2 is a diagrammatic elevation view of a spinal column segment showing one embodiment insertion instrument mounted to anchor assemblies engaged to adjacent vertebrae and a connecting element before insertion through the anchor assemblies.

FIG. 3 is the view of FIG. 2 with the connecting element inserted through the anchor assemblies.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Devices and methods for facilitating placement of a connecting element between anchor assemblies are provided for spinal surgical procedures. The connecting element is positioned in the patient along a connecting element insertion axis. The insertion axis extends between receivers of the anchor assemblies. The receivers of the anchor assemblies each include a pair of arms defining a passage therebetween for receiving the connecting element. The leading ends of the arms are oriented in the direction from which the connecting element is inserted into the passage. The leading ends of the arms include chamfered surfaces adjacent the passage to facilitate endwise insertion of the connecting element into the passage as the connecting element is moved along the insertion axis.

The anchor assemblies discussed herein can be multi-axial or uni-axial in form, and can include an anchor member engageable to a vertebra and a receiver for receiving a connecting element. The multi-axial anchor assemblies allow the anchor member coupled to the receiver to be positioned at various angles relative to the receiver. The uni-axial anchor assemblies provide a fixed positioning of the receiver relative to the anchor member. The anchor member of the anchor assemblies forms a distal lower portion that is engageable engageable to a vertebral body with the proximal receiver positioned adjacent the vertebra. In one embodiment, the anchor member is in the form of a bone screw with a threaded shaft and a proximal head that is pivotally captured in the receiver. In other embodiments, the distal anchor member can be in the form of a hook, staple, cable, tether, suture anchor, interbody fusion implant, artificial disc implant, bolt, or other structure engageable to bony tissue. The receiver defines a passage that receives a connecting element, such as a rod, tether, wire, cable, plate or other elongated linking member that can extend between one or more additional anchor assemblies secured to one or more additional vertebrae.

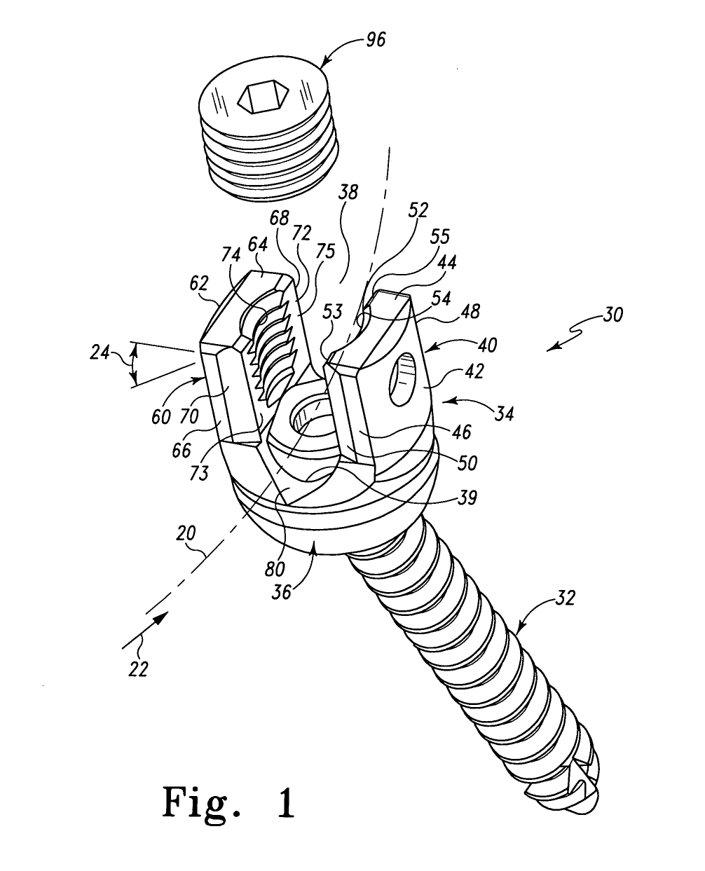

In FIG. 1, anchor assembly 30 includes an anchor member 32 engageable to bony tissue and a receiver 34 for engagement with a connecting element. In one embodiment, anchor member 32 of anchor assembly 30 includes a threaded shaft to threadingly engaging bony tissue. The threaded shaft can be provided with self-drilling and/or self-tapping thread profiles to facilitate insertion into bony tissue. In another embodiment, the threaded shaft is configured for insertion in a pre-drilled and pre-tapped hole in the vertebral body.

In the illustrated embodiment, anchor member 32 is a pedicle screw. Receiver 34 can be in the form of a U-shaped saddle having a receiver to receive a connecting element positionable along the spinal column, such as connecting element 100. Receiver 34 is pivotal relative to anchor member 32, and anchor member 32 is pivotally captured in receiver 34. For example, anchor member 32 can be provided with an enlarged head at a proximal or upper end thereof that is pivotally captured in a bowl portion 36 forming a distal portion of receiver 34. Bowl portion 34 includes a distally oriented opening through which anchor member 32 extends, and defines a receptacle in which the head of anchor member 32 is pivotally captured. When anchor member 32 is engaged to the bony tissue, receiver 34 can be pivotally adjusted and repositioned as needed for engagement with connecting element 80. In another embodiment, receiver 34 is integral with and formed as a single piece with the anchor member 32, providing a uni-axial anchor 30. Furthermore, a set screw, washer, crown, cap or other device may be provided for engagement within and/or about receiver 34 to secure connecting element 100 thereto.

Receiver 34 includes a first arm 40 and a second arm 60 extending proximally from bowl portion 36. A passage 38 extends between arms 40, 60, and connecting element insertion axis 20 extends through passage 38. First arm 40 includes an outer surface 42 extending from bowl portion 36 to a proximal end surface 44. Outer surface 42 further extends in the direction defined by insertion axis 20 between a leading end surface 46 and a trailing end surface 48. End surfaces 46, 48 extend generally orthogonally to insertion axis 20. Leading end surface 46 is oriented toward the direction from which the connecting element will be positioned for endwise insertion through passage 38, the insertion direction being indicated by arrow 22. A chamfered surface 50 extends from leading end surface 46 to an internal surface 52. Internal surface 52 includes a concavely curved central portion 54 defining a thread profile to threadingly engage a coupling member 96 to engage connecting element 100 in passage 38. Internal surface 52 further includes a first linear portion 53 extending between chamfered surface 50 and central portion 54 and a second linear portion 55 extending between trailing end surface 48 and central portion 54.

Second arm 60 includes an outer surface 62 extending from bowl portion 36 to a proximal end surface 64. Outer surface 62 further extends in the direction defined by insertion axis 20 between a leading end surface 66 and a trailing end surface 68. End surfaces 66, 68 extend generally orthogonally to insertion axis 20. Leading end surface 66 is oriented toward the direction from which the connecting element will be positioned along insertion axis 20 through passage 38, the insertion direction being indicated by arrow 22. A chamfered surface 70 extends from leading end surface 66 to an internal surface 72. Internal surface 72 includes a concavely curved central portion 74 defining a thread profile to threadingly engage a coupling member 98 to engage connecting element 100 in passage 38. Internal surface 72 further includes a first linear portion 73 extending between chamfered surface 70 and central portion 74 and a second linear portion 75 extending between trailing end surface 68 and central portion 74.

Leading end surfaces 46, 66 are orthogonal to or at least substantially perpendicular to insertion axis 20. Chamfered surfaces 50, 70 are angled toward internal surfaces 52, 72 to provide a smooth or less abrupt transition from leading end surfaces 46, 66 into passage 38. In this configuration, endwise insertion of the connecting element into passage 38 along insertion axis 20 from the direction indicated by arrow 22 is facilitated. For example, the leading end 102 of connecting element 100 may contact one of the chamfered surfaces 50, 70 as it is inserted. The chamfered surfaces 50, 70 in turn direct the connecting element 100 into passage 38 so that insertion may continue in a smooth and controlled manner without arms 40, 60 blocking insertion of the connecting element, or without the connecting element causing receiver 34 to pivot so that passage 38 is out of alignment with insertion axis 20.

In the illustrated embodiment, trailing end surfaces 48, 68 extend to internal surfaces 52, 72 without a chamfered surface. In another embodiment, chamfered surfaces are provided between trailing end surfaces 48, 68 and the respective internal surface, facilitating endwise insertion of the connecting element into passage 38 from the opposite direction. For embodiments providing leading end surfaces and/or trailing end surfaces orthogonally oriented to the insertion axis, connection of the receiver to anchor extensions and other instruments is facilitated, and a robust design of the receiver can be provided to resist loading exerted on the receiver.

Receiver 34 further includes a ramped surface 80 extending between bowl portion 36 and a bottom surface 39 of passage 38 extending between arms 40, 60. In the illustrated embodiment, bottom surface 39 is interrupted by crown 90. Other embodiments contemplate a continuous or substantially continuous bottom surface 39. Ramped surface 80 facilitates placement and guiding of connecting element 100 through passage 38 should leading end 102 of connecting element 100 contact receiver 34 below passage 38 along bowl portion 36.

In the illustrated embodiment, anchor assembly 30 includes crown 90 positioned in bowl portion 36 adjacent passage 38. Crown 90 includes a central opening 92 to receive a driving instrument (not shown) to apply a driving force to anchor member 32. When connecting element 100 is positioned in passage 38, it can be secured to receiver 34 with a coupling member 96, such as a set screw, threadingly engaged to internal surfaces 52, 72 along arms 40, 60. Coupling member 96 forces connecting element 100 in contact with crown 90, which in turn is forced into engagement with the head of anchor member 32 and secures anchor member 32 in position relative to receiver 34. The head of the anchor member 32 can be axially restrained in bowl portion 36 with a c-shaped retaining clip (not shown), an internal flange, or other suitable structure to prevent anchor member 32 from separating from receiver 34. Other embodiments contemplate other configurations for anchor assembly 30 as discussed herein, including those not including crown 90. In another embodiment, the coupling member could be internally threaded and engaged to external threads along arms 40, 60. In another embodiment, the coupling member includes multiple components engageable internally and/or externally to arms 40, 60.

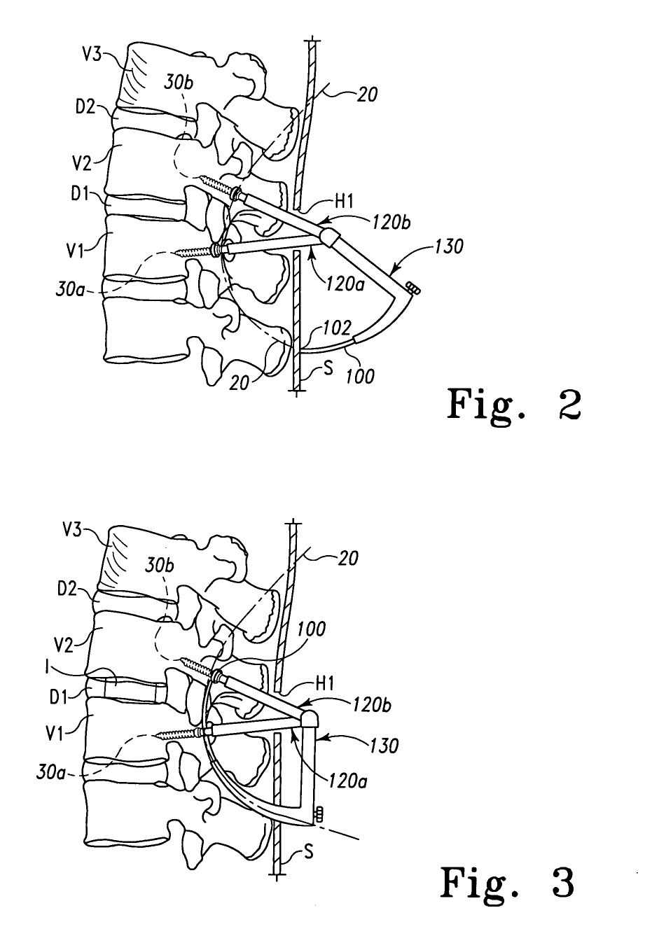

Referring now to FIGS. 2-3, one embodiment insertion technique for positioning a connecting element 100 through a pair of anchor assemblies 30a, 30b will be discussed. The spinal column segment is shown with three vertebrae V1, V2, V3. Disc space D1 is located between vertebrae V1 and V2, and disc space D2 is located between vertebrae V2 and V3. A first anchor assembly 30a is engaged to vertebra V1 and a second anchor assembly 30b is engaged to vertebra V2. A first extender 120a is coupled to first anchor 30a, and a second extender 120b is coupled to second anchor 30b. For embodiments where anchors 30a, 30b have multi-axial capabilities, extenders 120a, 120b can be manipulated so that their proximal ends are adjacent one another for mounting of an inserter instrument 130.

For minimally invasive procedures, anchors 30a, 30b can be engaged to the respective vertebrae V1, V2 through percutaneous pathways formed through skin S and/or the tissue between skin S and the vertebrae. A separate pathway can be provided for each anchor or, as illustrated, a single pathway H1 is provided for both anchors. Connecting element 100 is mounted to inserter instrument 130, and can be guided along insertion axis 20 from a location outside the patient's body, as shown in FIG. 2, to a location extending between anchors 30a, 30b as shown in FIG. 3.

The receivers of anchors 30a, 30b are oriented so that leading end surfaces 46, 66 and chamfered surfaces 50, 70 face the direction from which connecting element 100 will be received for positioning in the passages of the receivers. The chamfered surfaces facilitate and guide insertion of connecting element 100 into the passages of the anchor assemblies as the leading end of connecting element 100 is guided through anchor assemblies 30a, 30b. In a minimally invasive approach to the vertebrae, visualization of the connecting element as it enters the connecting element can be prevented or reduced by tissue, and slight misalignments of the connecting element with the passages of the anchor assemblies can be corrected by the chamfered surfaces.

In the illustrated embodiment, connecting element 100 is moved in the caudal to cephalad direction. It is contemplated that the connecting element can be inserted in the cephalad to caudal direction, in the medial-lateral directions for transverse connectors, or in any other direction suitable for placement of a connecting element in a spinal procedure. Furthermore, the connecting element can be engaged to more than two anchor assemblies along the spinal column, and provide stabilization for multiple spinal motion segments. The connecting element can also be positioned for engagement with a single anchor assembly engaged to the spinal column, or for procedures involving single vertebra.

Connecting element 100 can be a spinal rod connectable to one or more anchor assemblies to rigidly stabilize the spinal column. Connecting element 100 can also be flexible to allow motion of the spinal motion segment or segments to which it is attached. Other embodiments contemplate that connecting element 100 can comprise multiple components. In another embodiment, the connecting element includes a carrier for a non-rigid implant such as a tether, and the carrier is employed to facilitate placement of the non-rigid connecting element through the anchor assembly in endwise fashion. Other embodiments contemplate other forms for the connecting element, including plates, wires, struts, cables, and other devices capable of endwise insertion through the receiver of the anchor assembly, either alone or via a carrier.

In one procedure, anchor assembly 30 can be inserted through a minimally invasive access portal for engagement of anchor member 32 with bony tissue of a vertebra, such as the pedicle of the vertebra. The minimally invasive access portal can be provided by a micro-incision, a sleeve, a sleeve with an expandable working channel, a retractor blade, or two or more retractor blades of a retractor system. Anchor assembly 30 can be guided to position anchor member 32 in a desired trajectory or path into the vertebra using fluoroscopic imaging, endoscopic viewing, or other suitable viewing or imaging systems. Anchor member 32 can be engaged to the vertebra by positioning a driving instrument through passage 38 and into engagement with anchor member 32 in bowl portion 36. The procedure can be repeated for one or more additional anchor assemblies engaged to the same vertebrae, or to different vertebrae, along one or more spinal motion segments.

In one procedure, extenders can be mounted to anchor assemblies 10 either before or after engagement of anchor member 32 to the adjacent vertebrae. The extenders extend from the anchor member to a proximal end located outside the patient. An insertion instrument for delivering the connecting element along the insertion axis to the anchor assemblies is provided that can be mounted to the proximal end of one or more of the extenders. The inserter is operable to pivot about the proximal ends of the extenders to deliver the connecting element along the insertion axis and through the passage of one or more of the receivers of the anchor assemblies.

Other embodiments for procedures contemplate other insertion techniques for the connecting element, including free hand insertion of the connecting element along the insertion axis, insertion instruments that are not coupled to the anchor assemblies; insertion instruments employing image-guided navigation system; insertion instruments that deliver the connecting element through tissue without retraction or cutting of the tissue; and insertion instruments that deliver the connecting element through open incisions where skin and tissue are retracted to accommodate placement of the connecting element along the insertion axis, for example.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. An anchor assembly for engaging a connecting element along the spinal column, comprising:
an anchor member engageable to a vertebral body; and
a receiver at a proximal end of said anchor member, said receiver including a first arm and a second arm each including a proximal end surface and an internal surface, said first and second arms each further including a leading end surface and an opposite trailing end surface extending distally from said proximal end surface thereof, said internal surfaces defining a passage between said arms extending along a connecting element insertion axis oriented in the direction from said leading end surfaces toward said trailing end surfaces, said leading end surfaces each being generally orthogonally oriented to said insertion axis, each of said first and second arms further comprising a chamfered surface extending from said leading end surface to said internal surface to facilitate endwise insertion of the connecting element into said passage.

2. The assembly of claim 1, wherein said trailing end surfaces are each generally orthogonally oriented to said insertion axis.

3. The assembly of claim 2, wherein each of said trailing end surfaces extends to said internal surface of said respective arm.

4. The assembly of claim 2, wherein each arm includes a chamfered surface extending between said internal surface and said trailing end surface thereof.

5. The assembly of claim 1, wherein said chamfered surface of each of said first and second arms forms an angle with said leading end surface thereof, said angle being in the range from 30 degrees to 60 degrees.

6. The assembly of claim 1, wherein said receiver further includes a distal bowl portion and said pair of arms extending proximally from said bowl portion, said bowl portion pivotally receiving a proximal end of said anchor member therein.

7. The assembly of claim 6, wherein said anchor member is a bone screw.

8. The assembly of claim 6, wherein said receiver includes a bottom surface extending between said internal surfaces of said first and second arms, said receiver further including a ramped surface extending from said bottom surface to an external surface about said bowl portion.

9. The assembly of claim 1, wherein said internal surfaces each include a concavely curved central portion defining a thread profile extending therealong.

10. The assembly of claim 9, wherein said internal surfaces each include a first linear surface portion extending from said central portion to said chamfered surface and a second linear surface profile extending from said central portion to said trailing end surface, said first and second linear surface portions each extending generally parallel to said insertion axis.

11. A spinal stabilization system, comprising:
an elongated connecting element positionable along a spinal column, said connecting element extending between a leading end and a trailing end;
an anchor assembly for receiving said connecting element, said anchor assembly including:
an anchor member engageable to the spinal column; and
a receiver extending proximally from the anchor member, said receiver defining a passage for receiving said connecting element, said receiver including a first arm and a second arm each including a proximal end surface and an internal surface, said first and second arms each further including a leading end surface and an opposite trailing end surface extending distally from said proximal end surface along said arm, said internal surfaces defining said passage between said arms, said connecting element being positioned into said passage along a connecting element insertion axis extending in a direction from said leading end surfaces toward said trailing end surfaces, each of said first and second arms further comprising a chamfered surface extending from said leading end surface to said internal surface thereof to facilitate endwise insertion of said connecting element into said passage.

12. The system of claim 11, wherein said leading end surfaces are each generally orthogonally oriented to said insertion axis.

13. The system of claim 11, wherein said trailing end surfaces are each orthogonally oriented to said insertion axis.

14. The assembly of claim 13, wherein each arm includes a chamfered surface extending between said internal surface and said trailing end surface.

15. The assembly of claim 11, wherein said receiver further includes a distal bowl portion and said pair of arms extend proximally from said bowl portion, said bowl portion pivotally receiving a proximal end of said anchor member therein.

16. The assembly of claim 15, wherein:
said anchor member is a bone screw; and
said connecting element is a spinal rod.

17. The assembly of claim 11, wherein said internal surfaces each include a concavely curved central portion defining a thread profile extending therealong.

18. The assembly of claim 17, wherein said internal surfaces each include a first linear surface portion extending from said central portion to said chamfered surface and a second linear surface profile extending from said central portion to said trailing end surface, said first and second linear surface portions each extending generally parallel to said insertion axis.

19. A method for stabilizing a spinal column segment, comprising:
engaging an anchor assembly to a vertebra of the spinal column, the anchor assembly comprising a receiver including a pair of arms defining a passage therebetween, the pair of arms defining a proximal end opening in communication with the passage, the passage extending along an insertion axis oriented between the pair of arms from the leading end surfaces toward the trailing end surfaces, the receiver further including chamfered surfaces between the leading end surfaces and the passage; and
inserting a connecting element along the insertion axis through the passage in a direction extending from the leading end surfaces to the trailing end surfaces, the chamfered surfaces guiding endwise insertion of the connecting element into the passage.

20. The method of claim 19, further comprising:
securing the elongated connecting element in the passage of the receiver member with a coupling member engaged in the proximal end opening of the receiver.

21. The method of claim 19, wherein the receiver is pivotally engaged to the vertebra.

22. The method of claim 19, wherein:
the leading end surfaces are orthogonally oriented to the insertion axis; and
the passage is defined by internal surfaces of the pair of arms, the internal surfaces each including a concavely curved central portion, a leading end portion, and a trailing end portion, the leading and trailing end portions extending along the passage generally parallel to the insertion axis.
